(19) United States
(12) Patent Application Publication
(10) Pub. No.: US 2019/0122756 A1
(43) Pub. Date: Apr. 25, 2019

(54) TECHNIQUES FOR CALCULATING A NOURISHMENT VALUE

(71) Applicant: Min Max Nutrition LLC, Henderson, NV (US)

(72) Inventor: Frank Paterrov, Henderson, NV (US)

(21) Appl. No.: 16/162,175

(22) Filed: Oct. 16, 2018

Related U.S. Application Data
(60) Provisional application No. 62/575,100, filed on Oct. 20, 2017.

(51) Int. Cl. G16H 20/60; G01N 33/02; G06F 17/30; G06F 3/0482

(52) U.S. Cl. CPC G16H 20/60; G06F 3/0482; G06F 17/3097; G01N 33/02

(57) ABSTRACT

A method for measuring nourishment value is described. In one example, the method includes determining a list of food items, receiving a selection of a first food item, determining a first nutrient and a second nutrient of the first food item, calculating a first nourishment value associated with the first food item, identifying a list of recommended nutrients associated with the user, upon receiving the selection of the first food item, determining at least one nutrient remaining in the list of recommended nutrients, calculating a second nourishment value associated with a second food item in the list of food items based on the first nourishment value, one or more nutrients of the second food item and the at least one nutrient remaining in the list of recommended nutrients and updating the list of food items based at least in part on the second nourishment value.

### CROSS REFERENCES

The present Application for Patent claims the benefit of U.S. Provisional Patent Application No. 62/575,100, by PATERROV, entitled “TECHNIQUES FOR CALCULATING A NOURISHMENT VALUE,” filed Oct. 20, 2017, assigned to the assignee hereof, and which is expressly incorporated herein.

### BACKGROUND

The use of computer systems and computer-related technologies continues to increase at a rapid pace. This increased use of computer systems has influenced the advances made to computer-related technologies. Computer systems have increasingly become an integral part of the business world and the activities of individual consumers. Computer systems may be used to carry out several business, industry, and academic endeavors.

Eating is a necessary part of life. The body needs fuel (e.g., food) to function and operate properly. This need for fuel is often expressed as a hunger urge which drives consumers to eat (e.g., consume calories). However, consumers eat for a variety of reasons other than satisfying hunger. For example, consumers may eat simply for the purpose of being social. Indeed, eating has become a social activity and food has become deeply intertwined with social interaction. This leads consumers to eat more when they are with others who are eating. Advertising has also led to an increase in the consumption of food. In addition to the many promotional reasons for eating, consumers may also use eating as a coping mechanism for dealing with negative emotion (e.g., stress, depression). As a result, consumers often overeat. Overeating, coupled with lifestyle behaviors that are increasingly sedentary, has led to epidemic numbers of consumers being overweight and/or obese.

These negative trends have resulted in increased attention to becoming health conscience. Approaches for becoming healthy (e.g., maintaining a healthy weight) include increasing the number of calories that are burned (through exercise, for example) and/or limiting the number of calories that are consumed, for example. By only tracking calories, however, consumers may often miss out on consuming essential nutrients. There exists a need for nutrition program techniques that accounts for nutritional values provided by foods.

### SUMMARY

The described techniques relate to improved methods, systems, devices, and apparatuses that support techniques for calculating nourishment values associated with food items. More specifically, the described methods relate to techniques that help users build a plan from real food that provides them with high levels of essential nutrients as well as conditionally essential nutrients. For example, the described systems relate to maintaining a database of food items and nutrients associated with each food item. The described techniques further relate to calculating a numerical value (e.g., nourishment value) associated with each food item based on the nutrients that it provides.

In some examples, the described techniques may further relate to calculating the nourishment value of a particular food item based on the nutrients present in that food item as well as prior food consumption of one or more users. More specifically, the nourishment value may be determined based on nutrients that the user is missing from his or her diet. In some examples, users are presented with a list of foods for creating a consumption plan. In some cases, the list of foods may be dynamically sorted by or otherwise adjusted based on nourishment values. In some examples, the nourishment values of all foods may change based on the nutrients provided by a food item previously selected by the user, the nutrients that the user is missing, and the nutrients provided by the remaining foods in the list.

According to at least one example, systems, methods, non-transitory computer readable media and related devices for measuring nourishment value are described. In one example, the system and method may include determining, using a processor, a plurality of food items from a database associated with the plurality of food items, the plurality of food items including a first food item, determining a first nutrient and a second nutrient of the first food item in the plurality of food items, calculating a first raw measurement of the first nutrient and a second raw measurement of the second nutrient for each of the plurality of food items, calculating an initial nourishment value associated with the first food item based on the first raw measurement and the second raw measurement, calculating a bioavailability modifier associated with the first food item and a second modifier associated with the first food item, updating the initial nourishment value associated with the first food item to an updated nourishment value associated with the first food item based on the initial nourishment value, the bioavailability modifier, and the second modifier, and storing the updated nourishment value in the database associated with the plurality of food items.

In one example, the system and method may include calculating the first raw measurement of the first nutrient in a first metric system, calculating the second raw measurement of the second nutrient in a second metric system, and standardizing the first raw measurement of the first nutrient and the second raw measurement of the second nutrient based on the first metric system and the second metric system. In some cases, calculating the initial nourishment value associated with the first food item is based on the standardizing.

In some examples, the system and method may further include calculating a bioavailability of the first nutrient if a predetermined amount of the first item were consumed by a human, calculating a bioavailability of the second nutrient if the predetermined amount of the first item were consumed by the human, calculating a biochemical response of the first nutrient if the predetermined amount of the first item were consumed by the human, and calculating a biochemical response of the second nutrient if the predetermined amount of the first item were consumed by the human. In some cases, updating the initial nourishment value associated with the first food item is based on the bioavailability modifier associated with the first nutrient and the bioavailability modifier associated with the second nutrient.

In some examples, the second modifier comprises at least one of a dehydration modifier, a consumption amount modifier, or a manual modifier. The system and method may further include determining a concentration of water in the first food item, comparing the concentration of water to a predefined threshold to determine whether the concentration of water satisfies the predefined threshold, and determining the dehydration modifier based on the comparing. In some cases, updating the initial nourishment value associated with the first food item is based on the dehydration modifier. In some cases, the second modifier comprises the dehydration modifier.

The system and method may further include determining a first consumption amount of the first food item and a second consumption amount of a second food item. In some cases, the first food item and the second food item include the first nutrient, determining that the first consumption amount is different than the second consumption amount, and determining the consumption amount modifier based on the first consumption amount, the second consumption amount, and determining that the first consumption amount is different than the second consumption amount. In some cases, the second modifier comprises the consumption amount modifier. In some cases, updating the initial nourishment value associated with the second food item is based on the consumption amount modifier, the updated nourishment value associated with the first food item being different than the updated nourishment value associated with the second food item.

In some examples, the first raw measurement of the first nutrient and the second raw measurement of the second nutrient are determined from a first food composition database and a second food composition database, the first food composition database being different than the second food composition database.

The system and method may further include determining a third nutrient in the first food item, determining that a third raw measurement of the third nutrient is unavailable in the first food composition database and the second food composition database, and determining the manual modifier based on the third raw measurement being unavailable. In some cases, updating the initial nourishment value associated with the first food item is based on the manual modifier. In some cases, the second modifier comprises the manual modifier.

In some examples, the system and method may further include receiving a selection of the first food item from a list of food items, retrieving the stored updated nourishment value associated with the first food item from the database associated with the plurality of food items, and transmitting, using a wireless communication protocol, the updated nourishment value associated with the first food item for display at an electronic device associated with a user.

Some examples relate to systems, methods, non-transitory computer readable media and related devices for measuring nourishment value at a first electronic device. In one example, the system and method may include determining a list of food items, transmitting, using a wireless communication protocol, the list of food items to a second electronic device, receiving a selection of a first food item from the list of food items, determining a first nutrient and a second nutrient of the first food item based on the selection, calculating a first nourishment value associated with the first food item based on a first raw measurement of the first nutrient, a second raw measurement of the second nutrient, and at least one modifier, calculating a second nourishment value associated with a second food item in the list of food items based on the first nourishment value, determining a second list of food items based on the second nourishment value associated with the second food item, updating the list of food items based on the second nourishment value, and transmitting the updated list of food items to the second electronic device.

In one example, the system and method may further include identifying a list of recommended nutrients associated with the user, upon receiving the selection of the first food item, determining at least one nutrient remaining in the list of recommended nutrients, and determining one or more nutrients of the second food item. In some cases, calculating the second nourishment value associated with a second food item is based on the first nourishment value, the one or more nutrients of the second food item and the at least one nutrient remaining in the list of recommended nutrients.

The system and method may further include determining a third food item in the list of food items, calculating a third nourishment value associated with the third food item, and determining that the third nourishment value is greater than the second nourishment value based on comparing the second nourishment value and the third nourishment value to a recommended nourishment value. In some cases, updating the list of food items comprises promoting the third food item over the second food item.

In some examples, the system and method may further include retrieving a user profile associated with the user, and determining the recommended nourishment value based on the user profile. In some examples, the system and method may further include determining a first group associated with the first food item, determining that a second group is associated with the second food item and the first group is associated with a third food item, and displaying the second food item in the updated list of food items and restricting a display of the third food item in the updated list of food items, based on the determining.

In some examples, the system and method may further include determining the first raw measurement of the first nutrient and the second raw measurement of the second nutrient in a predetermined amount of each food item in the list of food items. In some examples, the at least one modifier comprises at least one of a bioavailability modifier, a dehydration modifier, a consumption amount modifier, or a manual modifier.

The system and method may further include calculating a bioavailability of the first nutrient if a predetermined amount of the first item were consumed by a human, calculating a biochemical response of the first nutrient if the predetermined amount of the first item were consumed by the human, determining the bioavailability modifier associated with the first nutrient based on the bioavailability of the first nutrient and the biochemical response of the first nutrient, calculating a bioavailability of the second nutrient if the predetermined amount of the first item were consumed by the human, calculating a biochemical response of the second nutrient if the predetermined amount of the first item were consumed by the human, and determining the bioavailability modifier associated with the second nutrient based on the bioavailability of the second nutrient and the biochemical response of the second nutrient. In some cases, determining the first nourishment value is based on the bioavailability modifier associated with the first nutrient and the bioavailability modifier associated with the second nutrient. In some cases, the at least one modifier comprises the bioavailability modifier.

### BRIEF DESCRIPTION OF THE DRAWINGS

The accompanying drawings illustrate a number of exemplary examples and are a part of the specification. Together with the following description, these drawings demonstrate and explain various principles of the instant disclosure.

FIG. 1 is a block diagram illustrating one example of an environment in which the present systems and methods may be implemented;

FIG. 2 is a block diagram illustrating one example of a nourishment value component;

FIG. 3 is an exemplary user interface for a nourishment value calculator and food tracker;

FIG. 4 is a flow diagram illustrating one example of a method in accordance with various aspects of this disclosure;

FIG. 5 is a flow diagram illustrating one example of a method in accordance with various aspects of this disclosure;

FIG. 6 is a block diagram of a computer system for implementing the present systems and methods accordance with various aspects of this disclosure; and

FIG. 7 depicts a block diagram of a computer system for implementing the present systems and methods.

The examples described herein are susceptible to various modifications and alternative forms, specific examples have been shown by way of example in the drawings and will be described in detail herein. However, the exemplary examples described herein are not intended to be limited to the particular forms disclosed. Rather, the instant disclosure covers all modifications, equivalents, and alternatives falling within the scope of the appended claims.

### DETAILED DESCRIPTION

The systems and methods described herein relate to measuring nourishment value and tracking consumption of food items. More specifically, the systems and methods described herein relate to a tool (e.g., an application) that helps users build a diet from real foods that will provide them with high levels of all the essential nutrients and some conditionally essential nutrients. Essential nutrients are nutrients which the body cannot make. Conditionally essential nutrients are nutrients which the body can make, but their biosynthesis may be limited under certain conditions and are important for the human body to consume. The systems and methods described herein describes methods for assigning a nourishment value to a plurality of foods. The nourishment value may be a numerical value, and may be assigned to foods based on the nutrients it provides and the nutrients the user is missing.

The systems and methods described herein present users with a list of foods to choose from. The list of foods may be sorted by the nourishment value associated with each food. In some examples, the list of foods may be sorted from highest nourishment value to lowest nourishment value. The nourishment value of all foods may change based on the nutrients provided by a previous food selection, the nutrients that the user is still missing, and the nutrients provided by the remaining foods. The systems and methods also displays a food tracker to the users which helps users know when it is time to eat a type of food.

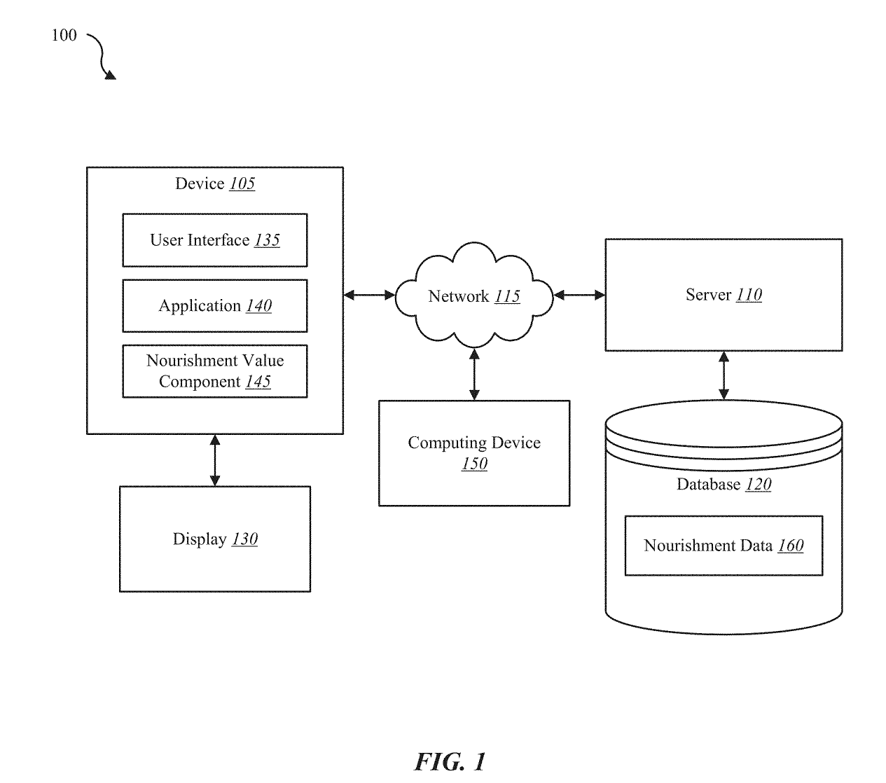

FIG. 1 is a block diagram illustrating one example of an environment 100 in which the present systems and methods may be implemented. In some examples, the systems and methods described herein may be performed on a device (e.g., device 105). As depicted, the environment 100 may include a device 105, server 110, a display 130, a computing device 150, and a network 115 that allows the device 105, the server 110, and the computing device 150 to communicate with one another.

Examples of the device 105 may include any combination of networking devices, mobile devices, smart phones, personal computing devices, computers, laptops, desktops, servers, media content set top boxes, or any combination thereof. Further examples of device 105 may include at least one of a network router, a wired router, a wireless router, network interface controller, wireless network interface controller, a modem, network bridge, network hub, network switch, a multilayer switch, a gateway, a bridge router, a multiplexor, a repeater, a firewall, a proxy server, or any combination thereof.

Examples of computing device 150 may include any combination of a mobile computing device, a laptop, a desktop, a server, a media set top box, or any combination thereof. Examples of server 110 may include at least one of a cloud application server, a remote cloud storage server, a data server, a cloud server, a server associated with an automation service provider, proxy server, mail server, web server, application server, database server, communications server, file server, home server, mobile server, name server, or any combination thereof.

In some configurations, the device 105 may include a user interface 135, application 140, and nourishment value component 145. Although the components of the device 105 are depicted as being internal to the device 105, it is understood that one or more of the components may be external to the device 105 and connect to device 105 through wired and/or wireless connections. In some examples, application 140 may be installed on computing device 150 in order to allow a user to interface with a function of device 105, nourishment value component 145, and/or server 110. In some cases, application 140 may include a software application such as an online application accessed via a web browser, a cloud based application, a web based application, a mobile application configured to access the Internet, a desktop application configured to access the Internet, or any combination thereof. In some cases, application 140 may be installed on an external device (not shown) connected to the device 150, and may allow a user to interface with a function of device 105, nourishment value component 145, and/or server 110.

In some examples, device 105 may communicate with server 110 via network 115. Examples of network 115 may include any combination of cloud networks, local area networks (LAN), wide area networks (WAN), virtual private networks (VPN), wireless networks (using 802.11, for example), cellular networks (using 3G and/or LTE, for example), etc. In some configurations, the network 115 may include the Internet. It is noted that in some examples, the device 105 may not include a nourishment value component 145. For example, device 105 may include application 140 that allows device 105 to interface with an external machine via nourishment value component 145 located on another device such as computing device 150 and/or server 110. In some examples, device 105, and server 110 may include a nourishment value component 145 where at least a portion of the functions of nourishment value component 145 are performed separately and/or concurrently on device 105, and/or server 110. Likewise, in some examples, a user may access the functions of device 105 (directly or through device 105 via nourishment value component 145) from computing device 150. For example, in some examples, computing device 150 includes a mobile application that interfaces with one or more functions of device 105, nourishment value component 145, and/or server 110.

In some examples, server 110 may be coupled to database 120. Database 120 may be internal or external to the server 110. In one example, device 105 may be coupled directly to database 120, database 120 being internal or external to device 105. Database 120 may include nourishment data 160. For example, nourishment data 160 may include information about nutrients associated with one or more food items.

Nourishment value component 145 may enable a computing device to determine a plurality of food items from a database associated with the plurality of food items, the plurality of food items including a first food item. In some cases, after determining the plurality of food items, the nourishment value component 145 may determine a first nutrient and a second nutrient of the first food item in the plurality of food items and calculate a first raw measurement of the first nutrient and a second raw measurement of the second nutrient for each of the plurality of food items.

The nourishment value component 145 may then calculate an initial nourishment value associated with the first food item based on the first raw measurement and the second raw measurement. After calculating the initial nourishment value, the nourishment value component 145 may calculate a bioavailability modifier associated with the first food item and at least a second modifier associated with the first food item. The nourishment value component 145 may update the initial nourishment value associated with the first food item to an updated nourishment value associated with the first food item based on the initial nourishment value, the bioavailability modifier, and the second modifier. The nourishment value component 145 may then store the updated nourishment value in the database associated with the plurality of food items.

For example, the updated nourishment value may be stored in database 120. In some examples, nourishment value component 145 may be configured to perform the systems and methods described herein in conjunction with user interface 135 and application 140. User interface 135 may enable a user to interact with, control, and/or program one or more functions of nourishment value component 145. Further details regarding the nourishment value component 145 are discussed below.

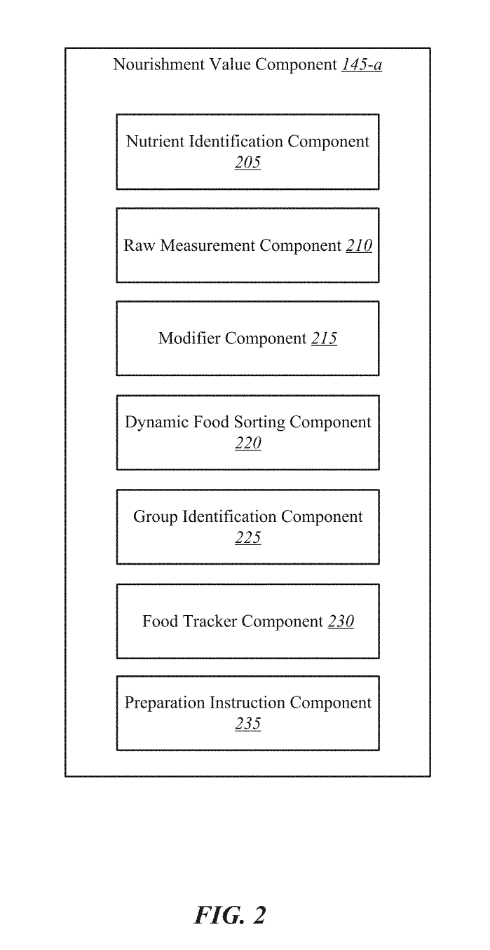

FIG. 2 is a block diagram illustrating one example of a nourishment value component 145-a. Nourishment value component 145-a may be one example of nourishment value component 145 depicted in FIG. 1. As depicted, nourishment value component 145-a may include nutrient identification component 205, raw measurement component 210, modifier component 215, dynamic food sorting component 220, group identification component 225, food tracker component 230, and preparation instruction component 235, among other aspects.

In some examples, at least a portion of nourishment value component 145 may be implemented in an application on a local machine (e.g., device 105 of FIG. 1), a remote computing device (e.g., computing device 150 of FIG. 1), a network device (e.g., network 115 of FIG. 1), a server (e.g., server 110 of FIG. 1), or any combination thereof.

The nourishment value component 145-a may perform techniques for calculating nourishment values associated with multiple food items. More specifically, the nourishment value component 145-a may generate a nourishment value associated with food items that helps users build a diet that provides them with high levels of essential nutrients as well as conditionally essential nutrients. For example, the nourishment value component 145-a may determine a plurality of food items from a database of food items and identify a plurality of nutrients associated with each food item. The nourishment value component 145-a may further calculate a numerical value (e.g., nourishment value) associated with each food item based on the nutrients that it provides. In some cases, a list of the food items may be dynamically sorted or adjusted according to the nourishment values, and may be presented to a user (e.g., before or after the dynamic sorting or adjusting). The nourishment value component 145-a, may dynamically sort the list of food items after receiving a selection of a food item from a user. In some cases, the nourishment value component 145-a may calculate the nourishment value of each food item based on the nutrients provided by a food item previously selected by the user, the nutrients that the user is missing, and the nutrients provided by the remaining foods in the list of food items.

In one example, nutrient identification component 205 may determine one or more nutrients associated with a food item. In some cases, the nutrients associated with the food item may be determined from multiple sources, such as scientific or health databases (e.g. relating to scientific papers, scientific journals, etc.). In some examples, the nutrient identification component 205 may identify a food item and may utilize a combination of databases to determine the nutrients present in the food item. In some examples, the one or more nutrients may include micro nutrients, macro nutrients, and super nutrients. In some examples, the micro nutrients may include vitamins and minerals. In some cases, the nutrient identification component 205 may be configured to determine one or more (e.g., at least thirteen) different types of vitamins.

For example, the vitamins identified by the nutrient identification component 205 may include: Vitamin A Retinol (or V01), Vitamin B-1 Thiamin (or V02), Vitamin B-2 Riboflavin (or V03), Vitamin B-3 Niacin (or V04), Vitamin B-5 Pantothenic Acid (or V05), Vitamin B-6 Pyridoxine (or V06), Vitamin B-7 Biotin (or V07), Vitamin B-9 Folate (or V08), Vitamin B-12 Cobalamin (or V09), Vitamin C Ascorbic Acid (or V10), Vitamin D Cholecalciferol (or V11), Vitamin E Alpha-Tocopherol (or V12), and Vitamin K Phylloquinone (or V13).

In some examples, the nutrient identification component 205 may be configured to determine one or more (e.g., at least fourteen) different types of minerals. For example, the minerals identified by the nutrient identification component 205 may include: Calcium Ca (or M01), Chloride Cl (or M02), Magnesium Mg (or M03), Phosphorus P (or M04), Potassium K (or M05), Sodium Na (or M06), Sulfur S (or M07), Chromium Cr (or M08), Copper Cu (or M09), Iodine I (or M10), Iron Fe (or M11), Manganese Mn (or M12), Selenium Se (or M13), and Zinc Zn (or M14).

In some cases, the macro nutrients may include fats, proteins and carbohydrates. In some examples, the nutrient identification component 205 may be configured to determine one or more (e.g., at least nine) different types of fats. For example, the fats may include F01 or Saturated Fatty Acid C12:0 Lauric Acid (dodecanoic acid), F02 or Polyunsaturated Fatty Acid C18:3n-3 Alpha-Linolenic Acid (cis-9,cis-12,cis-15-octadecatrienoic acid), F03 or Polyunsaturated Fatty Acid C20:5n-3 Eicosapentaenoic Acid (cis-5,cis-8,cis-11,cis-14,cis-17-eicosapentaenoic acid), F04 or Polyunsaturated Fatty Acid C22:6n-3 Docosahexaenoic Acid (cis-4,cis-7,cis-10,cis-13,cis-16,cis-19-docosahexaenoic acid), F05 or Polyunsaturated Fatty Acid C18:2n-6 Linoleic Acid (cis-9,cis-12-octadecadienoic acid), F06 or Polyunsaturated Fatty Acid C18:3n-6 Gamma-Linolenic Acid (cis-6,cis-9,cis-12-octadecatrienoic acid), F07 or Polyunsaturated Fatty Acid C20:4n-6 Arachidonic Acid (cis-5,cis-8,cis-11,cis-14-eicosatetraenoic acid), F08 or Monounsaturated Fatty Acid C16:1n-7 Palmitoleic Acid (cis-9-hexadecenoic acid), and F09 or Monounsaturated Fatty Acid C18:1n-9 Oleic Acid (cis-9-octadecenoic acid).

In some examples, the nutrient identification component 205 may be configured to identify one or more (e.g., at least nine) different types of proteins. As an example, the proteins identified by the nutrient identification component 205 may include Histidine (or P01), Isoleucine (or P02), Leucine (or P03), Lysine (or P04), Methionine (or P05), Phenylalanine (or P06), Threonine (or P07), Tryptophan (or P08), and Valine (or P09). In some examples, the nutrient identification component 205 may be configured to identify one or more (e.g., at least two) different types of carbohydrates. For example, the nutrient identification component 205 may identify C01 or Glucose, and C02 or Dietary Fiber.

In some examples, the super nutrients identified by the nutrient identification component 205 may include antioxidants and enzymes. For example, among antioxidants, the nutrient identification component 205 may identify A01 or Carotenoids and A02 or Flavonoids. In some examples, the nutrient identification component 205 may identify one or more (e.g., at least four) types of enzymes. As an example, the enzymes identified by the nutrient identification component 205 may include E01 or Fermentation, E02 or Amylases, E03 or Lipases, E04 or Proteases. In total, the nutrient identification component 205 may identify at least fifty three nutrients associated with food items.

In some implementations, raw measurement component 210 may be configured to calculate a raw measurement of a food item. In some cases, the raw measurement component 210 may receive one or more nutrients included in a food item. Upon receiving the identification of one or more nutrients, the raw measurement component 210 may calculate a raw measurement of the food item. More specifically, for a particular food item, the raw measurement component 210 may calculate a raw measurement value for each of the one or more (e.g., the fifty three) nutrients identified by the nutrient identification component 205. In one example, the nourishment value component 145-a may identify a first food item having food identification (ID) 1001 and a second food item having food ID 1002.

In such an example, the raw measurement component 210 may calculate a raw measurement value for the food item with food ID 1001 based on the nutrients present in the food item with food ID 1001. The nutrients present in food item with food ID 1001 may be nutrients V01 and V02. For example, the raw measurement component 210 may calculate a raw measurement score for food item with food ID 1001, such as 34 for nutrient V01 and 82 for Nutrient V02. In some cases, the raw measurement score may also be referred to as an initial Min Max Power of food with food ID 1001.

In some examples, the raw measurement component 210 may calculate a raw measurement value for the food item with food ID 1002 based on the nutrients present in the food item with food ID 1002. The nutrients present in food item with food ID 1002 may be nutrients V01 and V02. In some cases, the raw measurement component 210 may calculate a first raw measurement score for nutrient V01 and a second raw measurement score for nutrient V02. For example, the raw measurement component 210 may calculate the raw measurement score for food item with food ID 1002, such as 12 for nutrient V01 and 0 for Nutrient V02. In some cases, the raw measurement score may also be referred to as an initial Min Max Power of food with food ID 1002.

In some cases, the raw measurement component 210 may calculate the raw measurement for a food item using a measurement of nutrients present in the food item. In one example, the raw measurement component 210 may calculate an average of raw measurements per 100 g of food from scientific food composition databases. In some cases, the raw measurement component 210 may calculate a first raw measurement of a first nutrient in a first metric system and a second raw measurement of a second nutrient in a second metric system. The first metric system may be different from the second metric system.

Upon determining the measurements of the nutrients in different metric systems, the raw measurement component 210 may standardize the raw measurements. In some cases, the initial Min Max power values are derived based on examining the raw measurement of nutrients per the international standard of X/100 g of food obtained from scientific food composition databases. In some examples, the initial Min Max power values may be based on the Food and Agriculture Organization of the United Nations Food Composition Data Standards. In some examples, the raw measurements may be in grams/100 g of food, or milligrams/100 g of food, or micrograms/100 g of food. The raw measurement component 210 may be configured to standardize these differences in measurements into one numerical value system. In some implementations, when taking the raw measurement of nutrient content per 100 g of food, the results may vary and the averages are used. For example, if there are 3 raw measurements of grams of nutrient P01 per 100 g of Food ID 1009, 24 g, 25 g, and 26 g, the average of 25 g may be used.

In some cases, the raw measurement component 210 may be configured to examine all the raw measurements of a nutrient across all foods and uses one or more methods to assign the initial Min Max power values to each food item for each nutrient. This standardizes all the different gram, milligram, and microgram raw measurements into a standard numeric system. For example, the raw measurement component 210 may examine the raw measurements from multiple scientific nutrition food composition databases and use an algorithm to create a new numbering system to standardize and represent the values of nutrients in foods for all the essential nutrients and conditionally essential nutrients.

In some implementations, the raw measurement component 210 may calculate a total raw measurement score for a food item. The total raw measurement score of a food item may be based on the raw measurement scores of each nutrient present in the food item. For example, the raw measurement component 210 may determine a first total raw measurement score for food item with food ID 1001 and a second total raw measurement score for food item with food ID 1002. In some cases, the total raw measurement score may be referred to as a total initial Min Max power. In this example, the total initial Min Max Power for food with food ID 1001 is 116 and the total initial Min Max Power for food with food ID 1002 is 12.

In some examples, a total initial Min Max Power for a food with food ID 1001 may have a total initial Min Max power of 301, which is the sum of all 53 nutrient's initial Min Max power values for the food with food ID 1001.

In some implementations, modifier component 215 may be configured to calculate a plurality of modifiers associated with a food item. For example, the modifier component 215 may determine a bioavailability modifier, a dehydration modifier, a consumption amount modifier and a manual modifier. The modifiers to the initial raw measurement values (or initial Min Max power values) may be used to modify the initial raw measurement value of a food item to determine a modified measurement value of the food item.

In some examples, the modifier component 215 may calculate a bioavailability of a nutrient based on a predetermined portion of the food item being consumed by a human. For example, the modifier component may calculate a bioavailability of a nutrient if a predetermined amount (such as a predefined portion) of the food item were consumed by a human. The modifier component 215 may determine a biochemical response of the nutrient when the predetermined portion of the food item is consumed by a human.

More specifically, the modifier component 215 may calculate a biochemical response of the nutrient present in the food item, if the predetermined amount of the food item were consumed by the human. Nutrients may have different bioavailable and biochemical responses in the body depending on the type of food that provides them (e.g., animal vs. plant, raw vs. non-raw). For example, the nutrient M11 Iron comes in two major forms, heme iron and non-heme iron. On average, heme iron from animal meat is more bioavailable (absorbable) percentage wise than non-heme iron found in eggs, dairy, and plant based products. In some examples, scientific food composition databases generally measure or report both heme iron and non-heme iron the same way as if they were biochemically equivalent in absorption in the body.

In some implementations, the modifier component 215 may be configured to use the bioavailability modifier to boost the initial Min Max power values of a nutrient in a first food item and penalize the initial Min Max power values of the same nutrient in a second food item. For example, the modifier component 215 may boost the raw measurement value of Nutrient M11 Iron for all food items which are animal meat products that provide the Nutrient M11 Iron and penalize the raw measurement value of Nutrient M11 Iron values for all food items which are eggs, dairy, or plant based products that provide the Nutrient M11 Iron.

As an example, if the initial Min Max power value of chicken is 20 for the Nutrient M11, Iron Fe and the initial Min Max power value of spinach is also 20, the modifier component 215 may modify the initial Min Max power values to a modified Min Max Power value. Referring back to the example of chicken, the modifier component 215 may be configured to calculate a modified Min Max power value of chicken as 30 and a modified Min Max power value of 10 for spinach. This is because Iron is more bioavailable (such as absorbable) from chicken that it is from spinach.

As another example, the Nutrient V10, Vitamin C Ascorbic Acid is susceptible to losses when exposed to heat (i.e. cooking). In some examples, scientific food composition databases do not account for this loss to heat. As a result, the raw measurement value (or initial Min Max power) of raw foods which provide the Nutrient V10, Vitamin C Ascorbic Acid may be boosted and the raw measurement value (initial Min Max Power) of non-raw foods which provide the Nutrient V10, Vitamin C Ascorbic Acid may be penalized.

For example, if the initial Min Max power value of raw kiwi (raw measurement value of kiwi) is 20 for the Nutrient V10, Vitamin C Ascorbic Acid and the initial Min Max power value of non-raw cabbage is also 20 for the Nutrient V10, Vitamin C Ascorbic Acid, the modifier component 215 may implement the bioavailability modifier and may change the value of non-raw Cabbage from the initial Min Max power of 20 for Nutrient V10, Vitamin C Ascorbic Acid to the modified Min Max power of 10 for the Nutrient V10, Vitamin C Ascorbic Acid, since the Nutrient V10, Vitamin C Ascorbic Acid is partially lost when exposed to heat (i.e., when the cabbage was cooked).

In some implementations, modifier component 215 may determine the dehydration modifier based on concentration of water in a food item. In some examples, the dehydration modifier may also be referred to as a dried modifier. In some cases, the modifier component 215 may determine a concentration of water in a food item. Upon determining the concentration of water, the modifier component 215 may determine whether a particular food item may be considered as a dried food. The modifier component 215 may determine whether a dried food by comparing the concentration of water in a food item with a predefined threshold. For example, a food item may be considered dried if a water concentration level in the food item falls below the predetermined threshold.

In some examples, the dehydration modifier (or dried modifier) may be implemented for the measurements of dried foods. In some implementations, scientific food composition databases may include measurements taken from dried foods, such as lentils or kidney beans (such as food item that are usually not consumed dry). Generally, raw measurements of dried foods produce a higher concentration of nutrients than non-dried foods, due to the reduced water concentration.

In some examples, the modifier component 215 may account for the raw measurement of dried food items by penalizing the raw measurement value (or initial Min Max power value) for all food measurements that are taken from dried foods. For example, if lentils have an initial Min Max power value of 20 for the Nutrient P06, Phenylalanine, the dried modifier may reduce it to a modified Min Max power value of 15 for the Nutrient P06, Phenylalanine (for example) to compensate for the extra concentration of nutrients per 100 g of food taken during the measurement of a dried food.

In some implementations, the modifier component 215 may determine a standard consumption amount of a food item. The modifier component 215 may determine a consumption amount modifier based on the standard consumption amount of the food item. In some cases, the consumption amount modifier may also be referred to as a size modifier. In some cases, the modifier component 215 may determine the size modifier for the varying average serving size portions with foods. For example, gram for gram, the average person eats more grams of beef per serving on average than parsley per serving on average. To account for this difference in average consumption portions, the modifier component 215 may penalize the raw measurement value (or initial Min Max power value) of foods that are eaten in smaller serving sizes on average. As an example, all herbs may be penalized a predefined percentage (for example 25%) of their initial Min Max power values. In one example, if parsley has an initial Min Max power of 100 for Nutrient V03, Vitamin B-2 Riboflavin and an initial Min Max power of 200 for V04, Vitamin B-3 Niacin, then the consumption amount modifier (or size modifier) may change these values from an initial Min Max power of 100 for Nutrient V03, Vitamin B-2 Riboflavin to a modified Min Max power of 75 for Nutrient V03, Vitamin B-2 Riboflavin and an initial Min Max power value of 200 for V04, Vitamin B-3 Niacin to a modified Min Max power of 150 for V04, Vitamin B-3 Niacin to account for the smaller average serving size.

In some implementations, the modifier component 215 may identify a nutrient in a food item and may determine that a raw measurement of the nutrient is unavailable in food composition databases. In some examples, the modifier component 215 may receive the identification of the nutrient from nutrient identification component 205. In some examples, the modifier component 215 may receive an indication from the raw measurement component 210, indicating that the raw measurement of the nutrient is unavailable. In some examples, the modifier component 215 may determine a manual modifier based on the identified nutrient in the food item and the raw measurement of the nutrient being unavailable. For example, the manual modifier may be reserved for any special circumstance.

In some examples, there may be situations where scientific food composition databases do not contain data on some nutrients found in foods. In some examples, some scientific peer-reviewed journals may contain this information. For example, there may be insufficient data for the enzymes papain and bromelain (which are both part of the Nutrient E04, Proteases) in scientific food composition databases but scientific peer-reviewed journals may state that papaya contains high concentrations of the protease enzyme papain and pineapple contains high concentration of the protease enzyme bromelain.

In this situation, to accurately account for these enzymes in some foods (e.g., papaya and pineapple) which may missing from scientific food composition databases or whose data in unknown, the modifier component 215 may modify the initial Min Max power values for the Nutrient E04, Proteases. For example, the modifier component 215 may increase the initial Min Max power values for the Nutrient E04. For example, if the initial Min Max power values for the Nutrient E04, Proteases for papaya and pineapple are 10 and 20, the manual modifier may boost these to modified Min Max power values to 15 and 25 (for example) to account for the Proteases not accounted for in the raw measurements per 100 g of food found in scientific food composition databases.

Thus, in some examples, the average raw measurements per 100 g of food from scientific food composition databases for each nutrient in each food item may be converted to initial Min Max power values for each nutrient per food. The initial Min Max powers may then be multiplied by bioavailability modifiers, dried modifiers, size modifiers and manual modifiers which results in a modified Min Max power values for each nutrient per food.

Therefore, as an example, a modified Min Max power for food with food ID 1001 for Nutrient V01 may be 15, which may be calculated by multiplying the food ID 1001's initial Min Max power of 10 for Nutrient V01 times various modifiers which may be derived from scientific peer-reviewed journals. Also, a total modified Min Max power for food with food ID 1001 may be 348, which is the sum of all (e.g., fifty three) nutrient's modified Min Max power values for food with food ID 1001.

In some examples, dynamic food sorting component 220 may receive a selection of a food item from a list of food items. For example, a list of food items may be displayed to a user and the user may select a food item from the list. The dynamic food sorting component 220 may dynamically sort the remaining food items. In some examples, the dynamic food sorting component 220 may use a dynamic value (or total dynamic Min Max power value) to help the users to create a diet using the most efficient path with real foods to meet the program's nutrition requirements. In some examples, the dynamic food sorting component 220 may display seven gauges on a display associated with the user (one for each nutrient category: Vitamins, Minerals, Fats, Proteins, Carbohydrates, Antioxidants, and Enzymes). In some examples, each gauge may show a percentage from 0% to 100% indicating how much of each nutrient category's nutrition requirements the user has fulfilled with the foods they have selected. In some examples, the users may start with no foods selected. In such a case, all the seven gauges are at 0%. In some examples, when the user hovers or clicks on a nutrient category gauge, the dynamic food sorting component 220 may display the nutrients in that category and the fulfillment percentage of those nutrients. In some cases, the percentage may range from 0% to 100%. In some examples, a fulfillment percentage is shown for the category (e.g., Vitamins) and also for the nutrients in that category (e.g., Vitamin A, Vitamin C).

In some examples, the dynamic food sorting component 220 may receive a selection of a food item and may update the list of food items based on the modified raw measurement values of the remaining food items. In some examples, the dynamic food sorting component 220 may initiate a display of a list of foods that are available for the users to choose from and a list of foods they have selected. The available foods are sorted by order of total dynamic Min Max powers for each food item in the list of food items. from highest to lowest. This total dynamic Min Max power value may be the sum of all the dynamic Min Max power values that a single food provides.

In some cases, the dynamic food sorting component 220 may be configured to change the dynamic values based on the foods that a user has selected. Once a user selects a food, the nutrient category gauges may update their percentages to reflect the amount of nutrients provided by all the foods they have selected compared to the nutrition requirement for each nutrient. In some examples, each nutrient may have a Min Max power requirement that needs to be fulfilled entirely before it can be 100%. In some examples, the dynamic food sorting component 220 may dynamically resort the available food panel after each food is selected by a user. For example, the available food panel may be sorted using the dynamic Min Max power of each food from highest total dynamic Min Max power to lowest total dynamic Min Max power.

In some implementations, the dynamic food sorting component 220 may determine the dynamic Min Max power values of each nutrient and food item by evaluating how much Min Max power each nutrient still requires to be 100% fulfilled and how much modified Min Max power each food item provides for each nutrient. For example, if V01, Vitamin A Retinol has a Min Max power requirement of 120 and the user has only selected Liver, which provides a modified Min Max power of 115 for V01, Vitamin A Retinol, then the nutrient fulfillment percentage of V01, Vitamin A Retinol may be 95.83333% and may be rounded down to 95%.

In another example, if shrimp provides a modified Min Max power of 80 for V01, Vitamin A Retinol but the user only needs a Min Max power of 5 to complete the nutrition requirement for V01, Vitamin A Retinol, shrimp will then have a dynamic Min Max power of 5 for V01, Vitamin A Retinol instead of its modified Min Max power of 80. In such cases, the dynamic food sorting component 220 may assign the dynamic Min Max power to each of the remaining food items in the list of food items based on their modified Min Max power and the previous food selections of the user. This is done for each nutrient and each food, after each food selection. After the user selects one food, the available food list is sorted from highest to lowest by the total dynamic Min Max power values for each food.

In another example, if V01, Vitamin A Retinol has a Min Max Power requirement of 120 and the user has only selected butter which provides a modified Min Max power of 5 for V01, Vitamin A Retinol, then the dynamic food sorting component 220 may calculate a nutrient fulfillment percentage of V01, Vitamin A Retinol as 4.16% and round it down to 4%. In the same example, after the selection of butter, if shrimp provides a modified Min Max power of 80 for V01, Vitamin A Retinol and if the user still needs a Min Max power of 115 to complete the nutrition requirement for V01, Vitamin A Retinol, the dynamic food sorting component 220 may determine that shrimp has a full dynamic Min Max Power of 80 for V01, Vitamin A Retinol, which is the same as its original modified Min Max Power value of 80. This is done for each nutrient and each food after each food selection. After the user selects one food, the available food list is sorted from highest to lowest by the total dynamic Min Max power values for each food.

In some examples, the dynamic food sorting component 220 may use the following formula to determine a food with food ID 1001's dynamic Min Max power for Nutrient V01.

(Food ID 1001's dynamic Min Max power for Nutrient V01)=(Food ID 1001's modified Min Max power for Nutrient V01) IF (Food ID 1001's modified Min Max power for Nutrient V01) IS LESS THAN OR EQUAL TO (Nutrient V01's Min Max power requirement−Nutrient V01's Min Max power fulfillment) IF NOT THEN (Food ID 1001's dynamic Min Max power for Nutrient V01) IS EQUAL TO (Nutrient V01's Min Max power requirement−Nutrient V01's Min Max power fulfillment).

In some examples, the dynamic food sorting component 220 may calculate the total dynamic Min Max power value for each food taking the sum of at least fifty three dynamic Min Max power values for each nutrient. For example, the total dynamic Min Max power of food with food ID 1001 may be calculated as follows:

(Food ID 1001's total dynamic Min Max power)=(Food ID 1001's dynamic Min Max power for Nutrient V01)+(Food ID 1001's dynamic Min Max power for Nutrient V02)+(Food ID 1001's dynamic Min Max power for Nutrient V03)+(Food ID 1001's dynamic Min Max power for Nutrient V04)+(Food ID 1001's dynamic Min Max power for Nutrient V05)+(Food ID 1001's dynamic Min Max power for Nutrient V06)+(Food ID 1001's dynamic Min Max power for Nutrient V07)+(Food ID 1001's dynamic Min Max power for Nutrient V08)+(Food ID 1001's dynamic Min Max power for Nutrient V09)+(Food ID 1001's dynamic Min Max power for Nutrient V10)+(Food ID 1001's dynamic Min Max power for Nutrient V11)+(Food ID 1001's dynamic Min Max power for Nutrient V12)+(Food ID 1001's dynamic Min Max power for Nutrient V13)+(Food ID 1001's dynamic Min Max power for Nutrient M01)+(Food ID 1001's dynamic Min Max power for Nutrient M02)+(Food ID 1001's dynamic Min Max power for Nutrient M03)+(Food ID 1001's dynamic Min Max power for Nutrient M04)+(Food ID 1001's dynamic Min Max power for Nutrient M05)+(Food ID 1001's dynamic Min Max power for Nutrient M06)+(Food ID 1001's dynamic Min Max power for Nutrient M07)+(Food ID 1001's dynamic Min Max power for Nutrient M08)+(Food ID 1001's dynamic Min Max power for Nutrient M09)+(Food ID 1001's dynamic Min Max power for Nutrient M10)+(Food ID 1001's dynamic Min Max power for Nutrient M11)+(Food ID 1001's dynamic Min Max power for Nutrient M12)+(Food ID 1001's dynamic Min Max power for Nutrient M13)+(Food ID 1001's dynamic Min Max power for Nutrient M14)+(Food ID 1001's dynamic Min Max power for Nutrient F01)+(Food ID 1001's dynamic Min Max power for Nutrient F02)+(Food ID 1001's dynamic Min Max power for Nutrient F03)+(Food ID 1001's dynamic Min Max power for Nutrient F04)+(Food ID 1001's dynamic Min Max power for Nutrient F05)+(Food ID 1001's dynamic Min Max power for Nutrient F06)+(Food ID 1001's dynamic Min Max power for Nutrient F07)+(Food ID 1001's dynamic Min Max power for Nutrient F08)+(Food ID 1001's dynamic Min Max power for Nutrient F09)+(Food ID 1001's dynamic Min Max power for Nutrient P01)+(Food ID 1001's dynamic Min Max power for Nutrient P02)+(Food ID 1001's dynamic Min Max power for Nutrient P03)+(Food ID 1001's dynamic Min Max power for Nutrient P04)+(Food ID 1001's dynamic Min Max power for Nutrient P05)+(Food ID 1001's dynamic Min Max power for Nutrient P06)+(Food ID 1001's dynamic Min Max power for Nutrient P07)+(Food ID 1001's dynamic Min Max power for Nutrient P08)+(Food ID 1001's dynamic Min Max power for Nutrient P09)+(Food ID 1001's dynamic Min Max power for Nutrient C01)+(Food ID 1001's dynamic Min Max power for Nutrient C02)+(Food ID 1001's dynamic Min Max power for Nutrient A01)+(Food ID 1001's dynamic Min Max power for Nutrient A02)+(Food ID 1001's dynamic Min Max power for Nutrient E01)+(Food ID 1001's dynamic Min Max power for Nutrient E02)+(Food ID 1001's dynamic Min Max power for Nutrient E03)+(Food ID 1001's dynamic Min Max power for Nutrient E04).

After receiving a selection of a food item from a user, the dynamic food sorting component 220 may be configured to sort the available food panel from highest total dynamic Min Max power to lowest total dynamic Min Max power. In some examples, the list recalculates the total dynamic Min Max powers of each food item after a food selection.

In some examples, group identification component 225 may identify a first group associated with a first food item and a second group associated with a second food item. In one example, each food with a food ID is assigned a group ID. For example, beef liver, chicken liver, pork liver, and duck liver may all share the same group ID. In some cases, after receiving a selection of a food item, the group identification component 225 may adjust the display of a remaining list of food items based on the group IDs of each food item in the remaining list of food item. For example, if any of the foods in one group is selected, the remaining foods in that group will no longer appear under an available food panel for selection, even if they have not been selected and have a total dynamic Min Max power value greater than zero.

In some examples, the group identification component 225 may also assign a type associated with each food item. For examples, there may be three types, where a food item is associated with any one type. The three types may be Type 1, Type 2, and Type 3. In some examples, items belonging to Type 1 are not optional and do not provide any Min Max power (e.g., water). In some examples, items belonging to Type 2 are optional, but do not provide any Min Max power (e.g., sunshine). In some examples, items belonging to Type 3 are optional and provide Min Max power (e.g., kale).

In some examples, the group identification component 225 may automatically add Type 1 items (e.g., Water) to a user's selected foods upon account creation. In some examples, the Type 1 foods may not be removed from the user's selected foods. In some examples, the group identification component 225 may not automatically add Type 2 items (e.g., Sunshine) to the user's selected foods upon account creation. However, the user can add Type 2 items to their selected foods one at a time and remove Type 2 items from their selected foods one at a time. In some examples, the user may add or remove Type 2 items from his or her selected foods using a toggle bar on a toolbar displayed to the user. In some examples, the group identification component 225 may not add Type 3 items (e.g., Kale) to the user's selected foods upon account creation, but the user can add them to their selected foods from the available foods panel. In some examples, the user may add the Type 3 items one at a time.

In some examples, the group identification component 225 may prohibit users from removing these Type 3 items from their selected foods one at a time. Users may remove these Type 3 items from their selected foods all together at once using a start over button on the toolbar. In some examples, the users can only start over to remove all Type 3 items all together at once using the start over button on the toolbar a maximum of a predetermined number of times within a period. For example, the start over button may be used for a maximum of ten times per hour.

In some examples, the group identification component 225 may maintain a counter associated with the foods selected by a user. For example, users can select only a limited number of Type 3 items, which is set by the counter. In some cases, the group identification component 225 may display the counter value on the toolbar. Each time a user selects a Type 3 food, the group identification component 225 may be configured to reduce the counter value by 1. Once the counter reaches 0, the user cannot select any more Type 3 foods unless they start over.

In some implementations, food tracker component 230 may be configured to track the user's selected foods. In some cases, the food tracker component 230 may track food items to be consumed by the user. As an example, once the user is satisfied with their food selections and their nutrient fulfillment percentages (ideally at 100% nutrient fulfillment percentage for all nutrients), the user may select a food tracking option to help track when they should eat each food. In some implementations, food tracker component 230 is always available to the user, regardless of the number of foods that the user has selected. For example, the food tracker may be available if the user only has Type 1 water in their selected foods. In another example, the food tracker may be available if the user only has Type 1 water in their selected foods and Type 2 sunshine selected. In a further example, the food tracker may be available if the user only has type 1 water in their selected foods and any number of Type 3 foods selected.

Each food selected by the user may have a frequency assigned to it. This frequency value may be in hours, minutes, or days. In some examples, the food tracker component 230 may determine a frequency value for each food item selected by a user. In some examples, the frequency values are either every 12 hours, every 24 hours, every 48 hours, or every 96 hours. The food tracker component 230 may assign a frequency value to a particular food item. For example, a food with food ID 1001 may be every 12 hours while another food with food ID 1002 may be every 48 hours.

In some examples, the food tracker component 230 may sort one or more foods based on when each food is due. “Dueness” of a particular food item may include a time period after which the food item is due for consumption. In some cases, dueness of a food item may be based on a percentage that represents how much time has passed since the user last ate that food item. For example, if food item with food ID 1001 has a frequency of every 12 hours, the food tracker component 230 may calculate its dueness as follows after 0 hours, 6 hours, 12 hours, and 24 hours since last eating the food with food ID 1001. For example, 0 hours after eating food with food ID 1001—its dueness may be 0% when a frequency is every 12 hours. Similarly, 6 hours after eating the food with food ID 1001—its dueness may be 50% when the frequency is every 12 hours. 12 hours after eating the food with food ID 1001—its dueness may be 100% when the frequency is every 12 hours. And, 24 hours after eating the food with food ID 1001—its dueness may be 200% when the frequency is every 12 hours. In some cases, the food tracker component 230 may notify a user, indicating that a particular food item (food with food ID 1001 in this example) is due for consumption once the dueness is 100%. In some cases, the food tracker component 230 may sort each of the user's selected food items according to dueness from highest percentage to lowest percentage.

In some examples, the food tracker component 230 may be configured to identify an urgency group associated with a food item. In some cases, the urgency group of a food item may be based on a dueness of that food item. In some implementations, the food tracker component 230 may place each food item from the user's selected foods, into one of four urgency groups.

The four urgency groups may be Overdue, Time to Eat, Coming Soon, and On Track. In some examples, the Overdue urgency group may be characterized using red color, the Time to Eat urgency group may be characterized using orange color, the Coming Soon urgency group may be characterized using yellow color, and the On Track urgency group may be characterized using green color. In one example, the Overdue urgency group may include foods with a dueness percentage greater than 199%. In some examples, the Time to Eat urgency group may include foods with a dueness percentage falling between 100% and 199%. In some examples, the Coming Soon urgency group may include foods with a dueness percentage ranging between 50% and 99%. In some cases, the On Track urgency group may include food with a dueness percentage ranging from 0% to 49%.

In some examples, the food tracker component 230 may set Type 1 items (e.g., water) to 50% dueness upon account creation of a user. In some cases, the food tracker component 230 may set a dueness of both Type 2 items (e.g., sunshine) and Type 3 items (e.g., kale) to 50% dueness when added to the user's selected foods, regardless of when foods of these types were last eaten. In some examples, the food tracker component 230 may receive a click of a user on a food. Upon receiving a click, the food tracker component 230 may mark the food as eaten and move it to the bottom of the list. In some cases, upon receiving an indication of food consumption, the food tracker component 230 may change the dueness of the food to 0%.

In some examples, the food tracker component 230 may be configured to display a warning bar displaying a user's total nutrition fulfillment percentage (e.g., 20%) across all nutrients if the user has not fulfilled 100% of all nutrient fulfillment percentages. In some cases, the food tracker component 230 may inform the users that they are missing some or all nutrition requirements. In some cases, the following warning message may be displayed to a user by the food tracker component 230: “The foods you have chosen only fulfill 20% of our program's general (non-prescribed) nutrition requirements. Please use the food planner to see what you are missing.”

In some implementations, the food tracker component 230 may be configured to round down the fulfillment percentages of nutrients and nutrient categories. For example, if nutrient V01 has a Min Max power requirement of 110 and a Min Max power fulfillment of 89, the food tracker component 230 may calculate the fulfillment percentage for nutrient V01 as 80.909%. Then, the food tracker component 230 may round the calculated number down to a final fulfillment percentage of 80.

In some cases, preparation instruction component 235 may be configured to determine preparation instructions for each food item. After determining the preparation instructions, the preparation instruction component 235 may display a food name, a food illustration, and preparation instructions associated with the food item. In some examples, the preparation instruction component 235 may display preparation instructions to instruct users on how to prepare each food for optimal quality and optimal bioavailability. For example, the preparation instruction component 235 may determine a way of consuming a food item such that the nutrients of that food item remain bioavailable.

In some examples, each food may have a plurality of preparation instructions. In such cases, the preparation instruction component 235 may display the plurality of preparation instructions on a Food Planner and Food Tracker on a display of a user (separated by commas). In some examples, these preparation instructions are assigned to each food and are the same for all users. In the following example, the true or false flag represents whether each preparation instruction is displayed to the user on a display associated with a user (e.g., on a display of a device associated with a user). More specifically, true represents displayed, false represents not displayed.

1, element, false

2, plant, false

3, animal, false

4, raw, true

5, sprouted, true

6, cold-pressed, true

7, unrefined, true

8, fresh, true

9, non-sponsored third party product, true

10, pasture-raised, true

11, grass-fed, true

12, unhomogenized, true

13, wild-caught, true

14, naturally fermented, true

15, unsweetened, true

16, presoaked, true

17, organic, true

18, stable cooking fat, true

In some examples, the preparation instruction component 235 may send notifications of new preparation instructions to a user. For example, the preparation instruction component 235 may determine a new preparation instruction for a particular food item. Upon determining the new preparation instruction, the preparation instruction component 235 may send a push notification to the user indicating that the new preparation instructions are available.

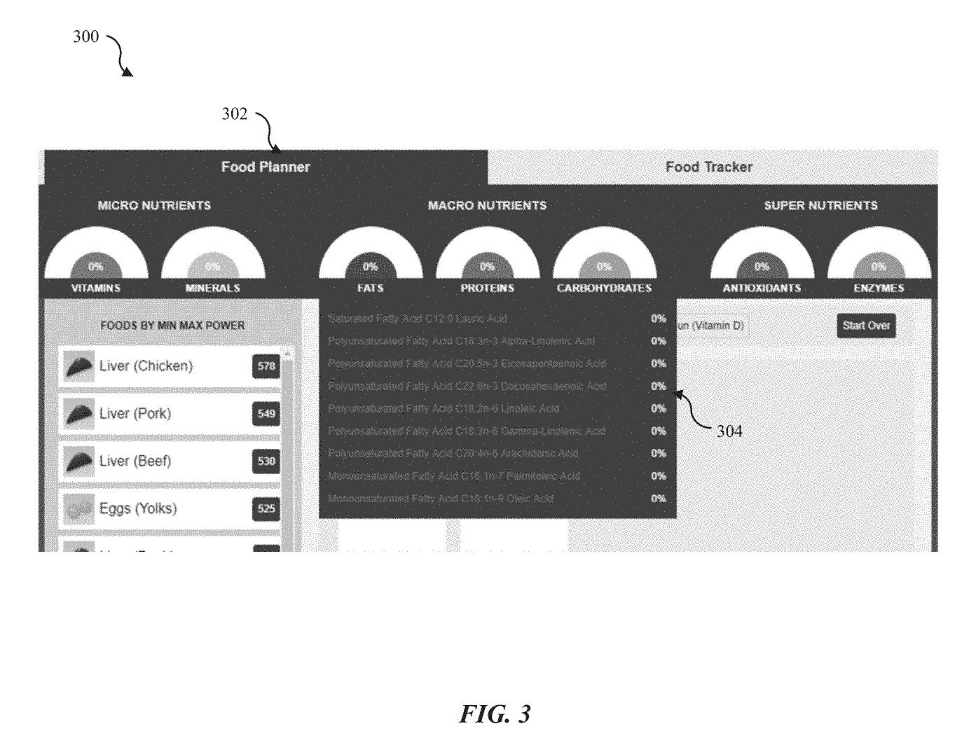

FIG. 3 describes an exemplary user interface 300 for a nourishment value calculator and food tracker is described. In some examples, the exemplary user interface may be generated by the nourishment value component 145, among other components or elements. The user interface shows a food planner section of a nourishment value calculating software. This may be generated when a user signs up for a nutrition program. The user may sign up for a nutrition program, with his or her personal information (address, home phone number, payment details, email address, password, mobile phone number, age, height, weight, sex).

For example, the user upon signing up may be presented with the exemplary user interface 300. When the user selects the food planner section 302, the user may be presented with three types of nutrients. The three types of nutrients may be micro nutrients, macro nutrients and super nutrients. As described with reference to FIG. 2, each type of nutrients may be divided into different types. As depicted in the example of FIG. 3, micro nutrients may be categorized into vitamins and minerals. Macro nutrients may be categorized into fats, proteins and carbohydrates. Also, super nutrients may be categorized into antioxidants and enzymes.

Upon selecting a type of nutrient (e.g., fat), the user may be presented with a detailed list 304. The user is also provided with a percentage counter for each type of nutrient. As described in the exemplary user interface 300, a list of food items sorted according to their nutritional values is presented to the user. Once the user selects any food item from the list of food items, the percentage counter for each type of nutrient may be updated based on the selection.

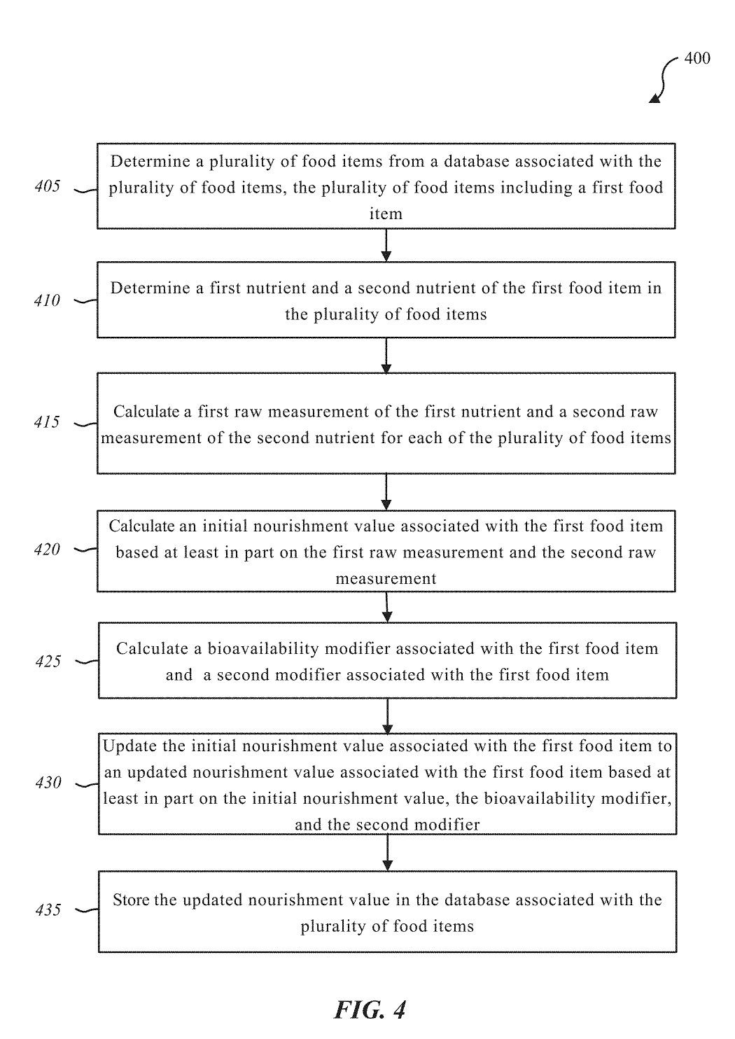

FIG. 4 is a flow diagram illustrating one example of a method 400 to calculate nourishment value. In some configurations, the method 800 may be implemented by the nourishment value component 145 illustrated in FIGS. 1, 2, and/or 3. In some configurations, the method 400 may be implemented in conjunction with device 105, server 110, network 115, database 120, components thereof, or any combination thereof

At block 405, method 400 may include determining a plurality of food items from a database associated with the plurality of food items, the plurality of food items including a first food item. At block 410, method 400 may include determining a first nutrient and a second nutrient of the first food item in the plurality of food items.

At block 415, method 400 may include calculating a first raw measurement of the first nutrient and a second raw measurement of the second nutrient for each of the plurality of food items. At block 420, method 400 may include calculating, an initial nourishment value associated with the first food item based at least in part on the first raw measurement and the second raw measurement.

At block 425, method 400 may include calculating a bioavailability modifier associated with the first food item and a second modifier associated with the first food item. At block 430, method 400 may include updating the initial nourishment value associated with the first food item to an updated nourishment value associated with the first food item based at least in part on the initial nourishment value, the bioavailability modifier, and the second modifier. At block 435, the method 400 may include storing the updated nourishment value in the database associated with the plurality of food items.

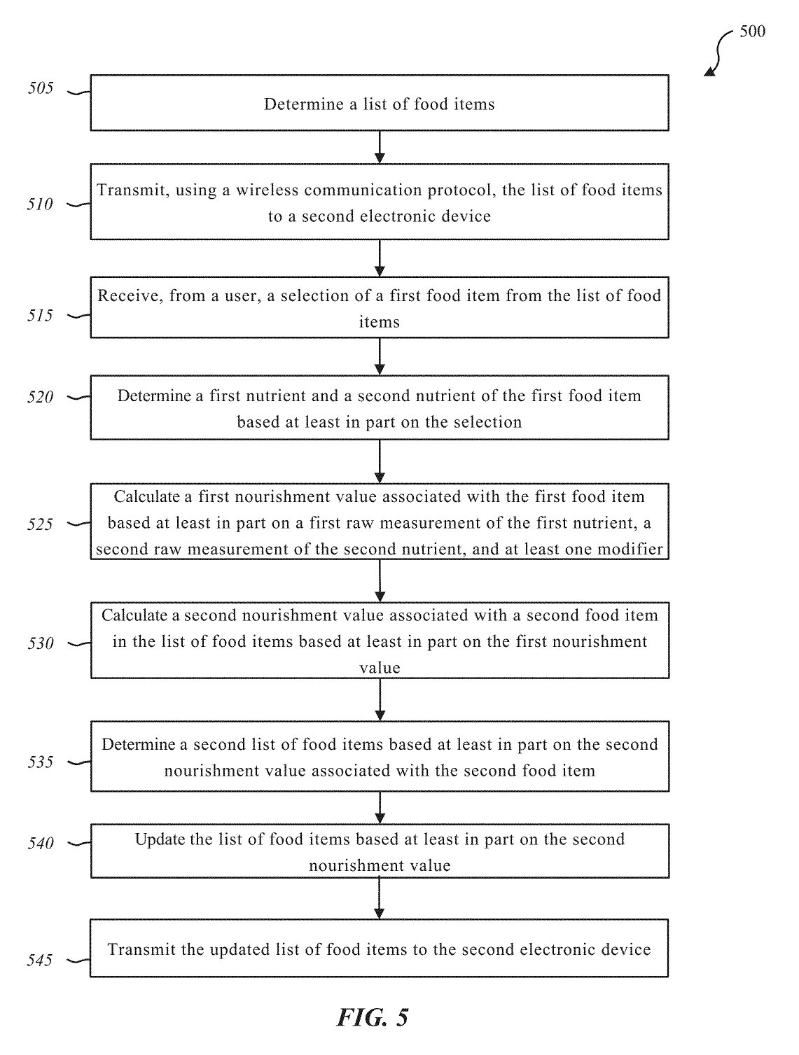

FIG. 5 is a flow diagram illustrating one example of a method 500 to calculate nourishment value. In some configurations, the method 500 may be implemented by the nourishment value component 145 illustrated in FIGS. 1, 2, 3, and/or 4. In some configurations, the method 500 may be implemented in conjunction with device 105, server 110, network 115, database 120, components thereof, or any combination thereof

At block 505, method 500 may include determining a list of food items. At block 510, method 500 may include transmitting, using a wireless communication protocol, the list of food items to a second electronic device. At block 515, method 500 may include receiving, from a user, a selection of a first food item from the list of food items.

At block 520, method 500 may include determining a first nutrient and a second nutrient of the first food item based at least in part on the selection. At block 525, method 500 may include calculating a first nourishment value associated with the first food item based at least in part on a first raw measurement of the first nutrient, a second raw measurement of the second nutrient, and at least one modifier. At block 530, method 500 may include calculating a second nourishment value associated with a second food item in the list of food items based at least in part on the first nourishment value.

At block 535, method 500 may include determining a second list of food items based at least in part on the second nourishment value associated with the second food item. At block 540, method 500 may include updating the list of food items based at least in part on the second nourishment value. At block 545, method 500 may include transmitting the updated list of food items to the second electronic device.

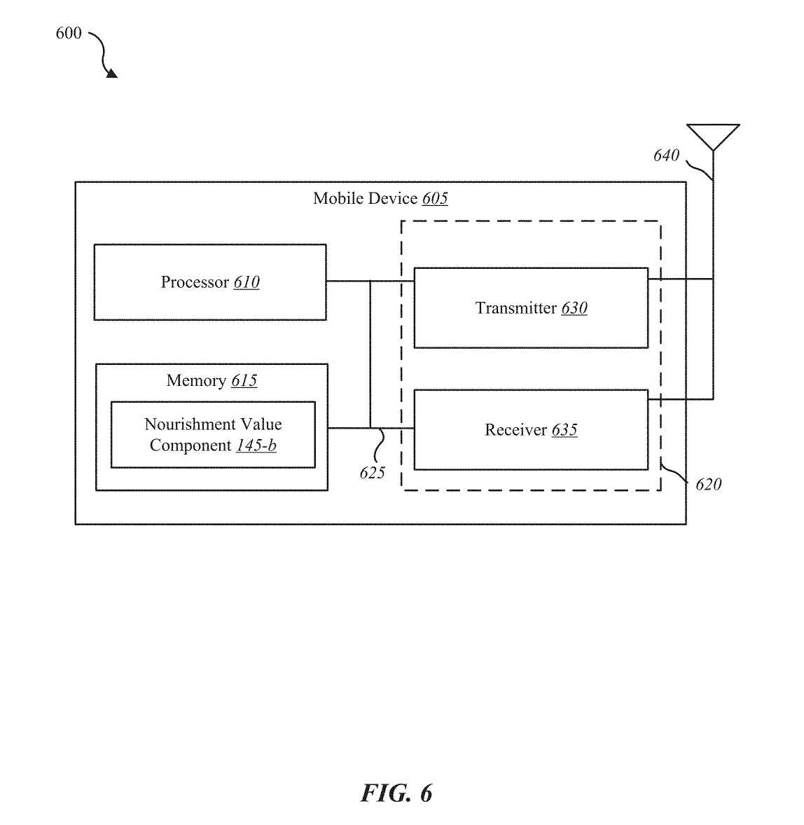

FIG. 6 depicts a block diagram of a computer system 600 suitable for implementing the present systems and methods. In one example, the computer system 600 may include a mobile device 605. The mobile device 605 may be an example of a device 105 depicted in FIG. 1. As depicted, the mobile device 605 includes a bus 625 which interconnects major subsystems of mobile device 605, such as a central processor 610, a system memory 615 (typically random access memory (RAM), but which may also include read-only memory (ROM), flash RAM, or the like), and a transceiver 620 that includes a transmitter 630, a receiver 635, and an antenna 640.

Bus 625 allows data communication between central processor 610 and system memory 615, which may include ROM or flash memory (neither shown), and RAM (not shown), as previously noted. The RAM is generally the main memory into which the operating system and application programs are loaded. The ROM or flash memory can contain, among other code, the Basic Input-Output system (BIOS) which controls basic hardware operation such as the interaction with peripheral components or devices.

For example, the nourishment value component 145-b to implement the present systems and methods may be stored within the system memory 615. The nourishment value component 145-b may be one example of the nourishment value component 145 depicted in FIGS. 1 and/or 2. Applications resident with mobile device 605 may be stored on and accessed via a non-transitory computer readable medium, such as a hard disk drive, an optical drive, or other storage medium. Additionally, applications can be in the form of electronic signals modulated in accordance with the application and data communication technology when accessed via a network.

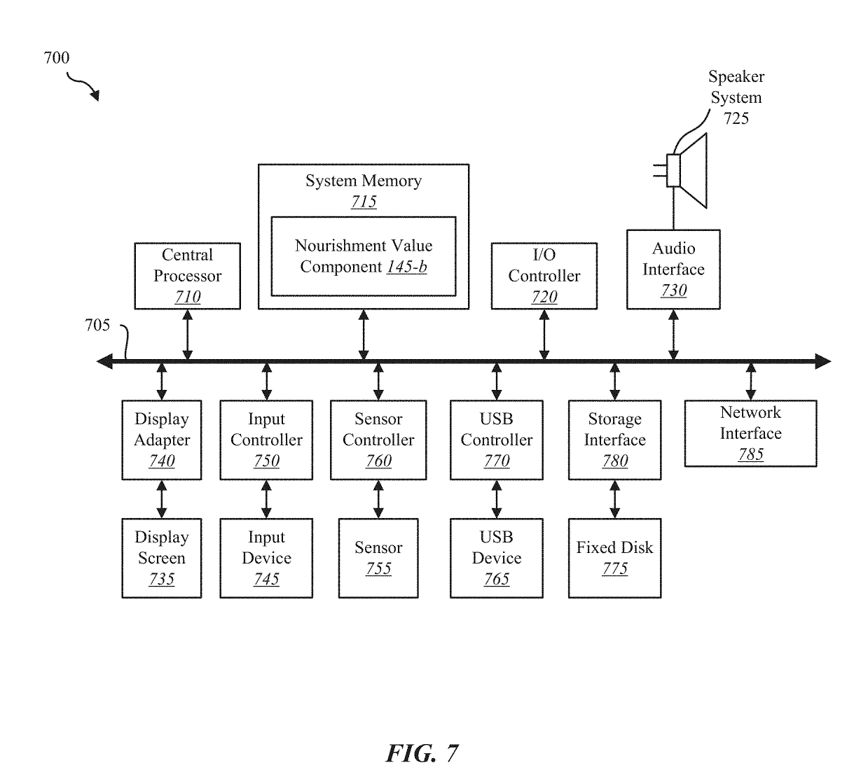

FIG. 7 depicts a block diagram of a computing device 700 suitable for implementing the present systems and methods. The device 700 may be an example of device 105, computing device 150, and/or server 110 illustrated in FIG. 1. In one configuration, device 700 includes a bus 705 which interconnects major subsystems of device 700, such as a central processor 710, a system memory 715 (typically RAM, but which may also include ROM, flash RAM, or the like), an input/output controller 720, an external audio device, such as a speaker system 725 via an audio output interface 730, an external device, such as a display screen 735 via display adapter 740, an input device 745 (e.g., remote control device interfaced with an input controller 750), multiple Universal Serial Bus (USB) devices 765 (interfaced with a USB controller 770), and a storage interface 780. Also included are at least one sensor 755 connected to bus 705 through a sensor controller 760 and a network interface 785 (coupled directly to bus 705).

Bus 705 allows data communication between central processor 710 and system memory 715, which may include ROM or flash memory (neither shown), and RAM (not shown), as previously noted. The RAM is generally the main memory into which the operating system and application programs are loaded. The ROM or flash memory can contain, among other code, the BIOS which controls basic hardware operation such as the interaction with peripheral components or devices. For example, the nourishment value component 145-c to implement the present systems and methods may be stored within the system memory 715. Applications (e.g., application 140) resident with device 700 are generally stored on and accessed via a non-transitory computer readable medium, such as a hard disk drive (e.g., fixed disk 775) or other storage medium. Additionally, applications can be in the form of electronic signals modulated in accordance with the application and data communication technology when accessed via interface 785.

Storage interface 780, as with the other storage interfaces of device 700, can connect to a standard computer readable medium for storage and/or retrieval of information, such as a fixed disk drive 775. Fixed disk drive 775 may be a part of device 700 or may be separate and accessed through other interface systems. Network interface 785 may provide a direct connection to a remote server via a direct network link to the Internet via a POP (point of presence). Network interface 785 may provide such connection using wireless techniques, including digital cellular telephone connection, Cellular Digital Packet Data (CDPD) connection, digital satellite data connection, or the like.

Many other devices and/or subsystems may be connected in a similar manner (e.g., entertainment system, computing device, remote cameras, wireless key fob, wall mounted user interface device, cell radio component, battery, alarm siren, door lock, lighting system, thermostat, home appliance monitor, utility equipment monitor, and so on). Conversely, all of the devices shown in FIG. 7 need not be present to practice the present systems and methods. The devices and subsystems can be interconnected in different ways from that shown in FIG. 7. The aspect of some operations of a system such as that shown in FIG. 7 are readily known in the art and are not discussed in detail in this application. Code to implement the present disclosure can be stored in a non-transitory computer-readable medium such as one or more of system memory 715 or fixed disk 775. The operating system provided on device 700 may be iOS®, ANDROID®, MS-DOS®, MS-WINDOWS®, OS/2®, UNIX®, LINUX®, or another known operating system.

Moreover, regarding the signals described herein, those skilled in the art will recognize that a signal can be directly transmitted from a first block to a second block, or a signal can be modified (e.g., amplified, attenuated, delayed, latched, buffered, inverted, filtered, or otherwise modified) between the blocks. Although the signals of the above described example are characterized as transmitted from one block to the next, other examples of the present systems and methods may include modified signals in place of such directly transmitted signals as long as the informational and/or functional aspect of the signal is transmitted between blocks. To some extent, a signal input at a second block can be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

The signals associated with device 700 may include wireless communication signals such as radio frequency, electromagnetics, local area network (LAN), wide area network (WAN), virtual private network (VPN), wireless network (using 802.11, for example), cellular network (using 3G and/or LTE, for example), and/or other signals. The network interface 785 may enable one or more of Wireless Wide Area Network (WWAN) (GSM, CDMA, and WCDMA), Wireless Local Area Network (WLAN) (including BLUETOOTH® and Wi-Fi), Wireless Metropolitan Area Network (WMAN) (WiMAX) for mobile communications, antennas for Wireless Personal Area Network (WPAN) applications (including Radio Frequency Identification (RFID) and Ultra-Wide Band (UWB)), etc.

The I/O controller 720 may operate in conjunction with network interface 785 and/or storage interface 780. The network interface 785 may enable device 700 with the ability to communicate with client devices (e.g., device 105 of FIG. 1), and/or other devices over the network 115 of FIG. 1. Network interface 785 may provide wired and/or wireless network connections. In some cases, network interface 785 may include an Ethernet adapter or Fiber Channel adapter. Storage interface 780 may enable device 700 to access one or more data storage devices. The one or more data storage devices may include two or more data tiers each. The storage interface 780 may include one or more of an Ethernet adapter, a Fiber Channel adapter, Fiber Channel Protocol (FCP) adapter, a Small Computer System Interface (SCSI) adapter, and iSCSI protocol adapter.

While the foregoing disclosure sets forth various examples using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered exemplary in nature since many other architectures can be implemented to achieve the same functionality.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

Further, while various examples have been described and/or illustrated herein in the context of fully functional computing systems, one or more of these exemplary examples may be distributed as a program product in a variety of forms, regardless of the particular type of computer-readable media used to actually carry out the distribution. The examples disclosed herein may also be implemented using software components that perform certain tasks. These software components may include script, batch, or other executable files that may be stored on a computer-readable storage medium or in a computing system. In some examples, these software components may configure a computing system to perform one or more of the exemplary examples disclosed herein.

The foregoing description, for purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the present systems and methods and their practical applications, to thereby enable others skilled in the art to best utilize the present systems and methods and various examples with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

What is claimed is:

1. A method for measuring nourishment value, comprising:
determining, using a processor, a plurality of food items from a database associated with the plurality of food items, the plurality of food items including a first food item;
determining, using the processor, a first nutrient and a second nutrient of the first food item in the plurality of food items;
calculating, using the processor, a first raw measurement of the first nutrient and a second raw measurement of the second nutrient for each of the plurality of food items;
calculating, using the processor, an initial nourishment value associated with the first food item based at least in part on the first raw measurement and the second raw measurement;
calculating, using the processor, a bioavailability modifier associated with the first food item and a second modifier associated with the first food item;
updating, using the processor, the initial nourishment value associated with the first food item to an updated nourishment value associated with the first food item based at least in part on the initial nourishment value, the bioavailability modifier, and the second modifier; and
storing, using the processor, the updated nourishment value in the database associated with the plurality of food items.

2. The method of claim 1, further comprising:
calculating the first raw measurement of the first nutrient in a first metric system;
calculating the second raw measurement of the second nutrient in a second metric system; and
standardizing the first raw measurement of the first nutrient and the second raw measurement of the second nutrient based at least in part on the first metric system and the second metric system, wherein calculating the initial nourishment value associated with the first food item is based at least in part on the standardizing.

3. The method of claim 1, wherein calculating the bioavailability modifier associated with the first food item comprises:
calculating a bioavailability of the first nutrient if a predetermined amount of the first item were consumed by a human;
calculating a bioavailability of the second nutrient if the predetermined amount of the first item were consumed by the human;
calculating a biochemical response of the first nutrient if the predetermined amount of the first item were consumed by the human; and
calculating a biochemical response of the second nutrient if the predetermined amount of the first item were consumed by the human, wherein updating the initial nourishment value associated with the first food item is based at least in part on the bioavailability modifier associated with the first nutrient and the bioavailability modifier associated with the second nutrient.

4. The method of claim 1, wherein the second modifier comprises at least one of a dehydration modifier, a consumption amount modifier, or a manual modifier.

5. The method of claim 4, further comprising:
determining a concentration of water in the first food item;
comparing the concentration of water to a predefined threshold to determine whether the concentration of water satisfies the predefined threshold; and
determining the dehydration modifier based at least in part on the comparing, wherein updating the initial nourishment value associated with the first food item is based at least in part on the dehydration modifier, and wherein the second modifier comprises the dehydration modifier.

6. The method of claim 4, further comprising:
determining a first consumption amount of the first food item and a second consumption amount of a second food item, wherein the first food item and the second food item include the first nutrient;
determining that the first consumption amount is different than the second consumption amount; and
determining the consumption amount modifier based at least in part on the first consumption amount, the second consumption amount, and determining that the first consumption amount is different than the second consumption amount, wherein the second modifier comprises the consumption amount modifier, and wherein updating the initial nourishment value associated with the second food item is based at least in part on the consumption amount modifier, the updated nourishment value associated with the first food item being different than the updated nourishment value associated with the second food item.

7. The method of claim 4, wherein the first raw measurement of the first nutrient and the second raw measurement of the second nutrient are determined from a first food composition database and a second food composition database, the first food composition database being different than the second food composition database.

8. The method of claim 7, further comprising:
determining a third nutrient in the first food item;
determining that a third raw measurement of the third nutrient is unavailable in the first food composition database and the second food composition database; and
determining the manual modifier based at least in part on the third raw measurement being unavailable, wherein updating the initial nourishment value associated with the first food item is based at least in part on the manual modifier, and wherein the second modifier comprises the manual modifier.

9. The method of claim 1, further comprising:
receiving a selection of the first food item from a list of food items;
retrieving the stored updated nourishment value associated with the first food item from the database associated with the plurality of food items; and
transmitting, using a wireless communication protocol, the updated nourishment value associated with the first food item for display at an electronic device associated with a user.

10. A method for measuring nourishment value at a first electronic device, comprising:
determining a list of food items;
transmitting, using a wireless communication protocol, the list of food items to a second electronic device;
receiving a selection of a first food item from the list of food items;
determining a first nutrient and a second nutrient of the first food item based at least in part on the selection;
calculating a first nourishment value associated with the first food item based at least in part on a first raw measurement of the first nutrient, a second raw measurement of the second nutrient, and at least one modifier;
calculating a second nourishment value associated with a second food item in the list of food items based at least in part on the first nourishment value;
determining a second list of food items based at least in part on the second nourishment value associated with the second food item;
updating the list of food items based at least in part on the second nourishment value; and
transmitting the updated list of food items to the second electronic device.

11. The method of claim 10, further comprising:
identifying a list of recommended nutrients associated with the user;
upon receiving the selection of the first food item, determining at least one nutrient remaining in the list of recommended nutrients;
determining one or more nutrients of the second food item; and
wherein calculating the second nourishment value associated with a second food item is based at least in part on the first nourishment value, the one or more nutrients of the second food item and the at least one nutrient remaining in the list of recommended nutrients.

12. The method of claim 10, further comprising:
determining a third food item in the list of food items;
calculating a third nourishment value associated with the third food item; and determining that the third nourishment value is greater than the second nourishment value based at least in part on comparing the second nourishment value and the third nourishment value to a recommended nourishment value, wherein updating the list of food items comprises promoting the third food item over the second food item.

13. The method of claim 12, further comprising:
retrieving a user profile associated with the user; and
determining the recommended nourishment value based at least in part on the user profile.

14. The method of claim 10, further comprising:
determining a first group associated with the first food item;
determining that a second group is associated with the second food item and the first group is associated with a third food item; and
displaying the second food item in the updated list of food items and restricting a display of the third food item in the updated list of food items, based at least in part on the determining.

15. The method of claim 10, further comprising determining the first raw measurement of the first nutrient and the second raw measurement of the second nutrient in a predetermined amount of each food item in the list of food items.

16. The method of claim 10, wherein the at least one modifier comprises at least one of a bioavailability modifier, a dehydration modifier, a consumption amount modifier, or a manual modifier.

17. The method of claim 16, further comprising:
calculating a bioavailability of the first nutrient if a predetermined amount of the first item were consumed by a human;
calculating a biochemical response of the first nutrient if the predetermined amount of the first item were consumed by the human;
determining the bioavailability modifier associated with the first nutrient based at least in part on the bioavailability of the first nutrient and the biochemical response of the first nutrient;
calculating a bioavailability of the second nutrient if the predetermined amount of the first item were consumed by the human;
calculating a biochemical response of the second nutrient if the predetermined amount of the first item were consumed by the human; and
determining the bioavailability modifier associated with the second nutrient based at least in part on the bioavailability of the second nutrient and the biochemical response of the second nutrient, wherein determining the first nourishment value is based at least in part on the bioavailability modifier associated with the first nutrient and the bioavailability modifier associated with the second nutrient, and wherein the at least one modifier comprises the bioavailability modifier.

18. An apparatus for measuring nourishment value, comprising:
a processor;
memory in electronic communication with the processor; and
instructions stored in the memory, the instructions being executable by the processor to:
determine a list of food items;
transmit, using a wireless communication protocol, the list of food items to a second electronic device;
receive a selection of a first food item from the list of food items;
determine a first nutrient and a second nutrient of the first food item based at least in part on the selection;
calculate a first nourishment value associated with the first food item based at least in part on a first raw measurement of the first nutrient, a second raw measurement of the second nutrient, and at least one modifier;
calculate a second nourishment value associated with a second food item in the list of food items based at least in part on the first nourishment value;
determine a second list of food items based at least in part on the second nourishment value associated with the second food item;
update the list of food items based at least in part on the second nourishment value; and
transmit the updated list of food items to the second electronic device.

19. The apparatus of claim 18, wherein the instructions are further executable by the processor to cause the apparatus to:
identify a list of recommended nutrients associated with the user;
upon receiving the selection of the first food item, determine at least one nutrient remaining in the list of recommended nutrients;
determine one or more nutrients of the second food item; and
wherein calculating the second nourishment value associated with a second food item is based at least in part on the first nourishment value, the one or more nutrients of the second food item and the at least one nutrient remaining in the list of recommended nutrients.

20. The apparatus of claim 18, wherein the instructions are further executable by the processor to cause the apparatus to:
determine a third food item in the list of food items;
calculate a third nourishment value associated with the third food item; and
determine that the third nourishment value is greater than the second nourishment value based at least in part on comparing the second nourishment value and the third nourishment value to a recommended nourishment value, wherein updating the list of food items comprises promoting the third food item over the second food item.
